(19) United States
(12) Patent Application Publication
(10) Pub. No.: US 2023/0263421 A1
(43) Pub. Date: Aug. 24, 2023

(54) ELECTRICAL IMPEDANCE TOMOGRAPHY BASED METHOD FOR FUNCTIONAL ELECTRICAL STIMULATION AND ELECTROMYOGRAPHY GARMENT

(71) Applicant: Battelle Memorial Institute, Columbus, OH (US)

(72) Inventors: Sam COLACHIS, Columbus, OH (US); David FRIEDENBERG, Columbus, OH (US)

(73) Assignee: Battelle Memorial Institute, Columbus, OH (US)

(21) Appl. No.: 18/018,468

(22) PCT Filed: Jul. 30, 2021
(86) PCT No.: PCT/US2021/043959

§ 371 Date: Jan. 27, 2023

Related U.S. Application Data
(60) Provisional application No. 63/058,984, filed on Jul. 30, 2020.

(51) Int. Cl. A61B 5/0536; A61N 1/04; A61N 1/36; A61B 5/389; A61B 5/296; A61B 5/256; A61B 5/00

(52) U.S. Cl. CPC A61B 5/0536; A61N 1/0484; A61N 1/0452; A61N 1/36031; A61N 1/36003; A61B 5/389; A61B 5/296; A61B 5/256; A61B 5/7267; A61B 5/6804; A61B 2560/0223; A61B 2562/0209; A61B 2562/04; A61N 1/0476

(57) ABSTRACT

Systems and methods which leverage electrical impedance tomography (EIT) for autonomous recalibration following garment donning are disclosed. The method may comprise performing an EIT measurement across an electrode array of an electrode garment and generating an anatomical model based on the EIT measurement. Next, one or more alignment variations may be estimated based on an alignment variation model. Finally, the electrode array is adjusted, automatically or manually, to accommodate the alignment variations using an alignment adjustment function.

### CROSS REFERENCE TO RELATED APPLICATIONS

This application claims the benefit of U.S. Provisional No. 63/058,984, filed on Jul. 30, 2020, which is incorporated by reference as if fully set forth.

### FIELD OF INVENTION

The invention relates generally to electrical impedance tomography (EIT). More particularly, this invention relates using EIT to calibrate an electrode garment.

### BACKGROUND

Limb paralysis is a common outcome of a spinal cord injury or stroke. Individuals with limb paralysis have hindered hand movement, making activities of daily living difficult to impossible. Neuromuscular electrical stimulation (NMES) uses electrical impulses to induce muscular contractions. Specifically, NMES comprises delivering electrical pulses via electrodes, through skeletal muscles, to activate a motor response. Muscle fibers in skeletal muscles respond to electrical signals sent through motor neurons. NMES induces a foreign electrical current which overrides the natural motor neuron activity and causes a muscle contraction. This may reanimate muscular movement in paralyzed limbs. NMES may also be used to enhance able limbs, for example, in sports performance enhancement and therapy. Functional electrical stimulation (FES) is a subset of NMES which focuses on promoting functional movement.

Electromyography (EMG) is a diagnostic test that measures how well the muscles respond to the electrical signals emitted to specialized nerve cells called motor nerves. In EMG garments, electrodes may be embedded in the garment to allow muscle excitation to be recorded.

A garment comprising an array of electrodes embedded therein may be configured for NMES, EMG, or both NMES and EMG. For example, the NeuroLife® group at the Battelle Memorial Institute has developed a high density NMES/EMG sleeve which both excites muscle and records muscle excitation and has a variety of applications. For example, the NeuroLife® sleeve may allow tetraplegic individuals to regain functional hand movements. The NeuroLife® sleeve may also be used as a component in a closed-loop system for rehab for stroke, spinal cord injury, multiple sclerosis, amyotrophic lateral sclerosis, or any other injuries that disrupt normal hand/arm function.

FES and/or EMG garments are susceptible to inter-session and inter- subject variability in electrode positioning during the donning process. Garment alignment inconsistencies and anatomical differences between subjects and/or users may affect system calibrations, such as FES patterns used to evoke movement. If the garment position is shifted, a corresponding shift in active electrodes may be required to compensate for the misalignment. Furthermore, anatomical differences between subjects and/or users may require de novo pattern calibration. Calibration may be achieved through trial and error where an operator manually selects individual electrodes for discrete activation and then iteratively refines the pattern. Not only is this process tedious and inefficient, but the discrete states of electrodes may impose a coarse resolution that make fine adjustments difficult. Therefore, a method for autonomous recalibration following garment donning would be extremely useful in the areas of NMES and EMG.

### SUMMARY

Systems and methods which leverage electrical impedance tomography (EIT) for autonomous recalibration following garment donning are disclosed. The method may comprise performing an EIT measurement across an electrode array of an electrode garment and constructing an anatomical model based on the EIT measurement. Next, one or more alignment variations may be estimated based on an alignment variation model. Finally, the electrode array is adjusted, automatically or manually, to accommodate the alignment variations using an alignment adjustment function.

### BRIEF DESCRIPTION OF THE DRAWINGS

FIG. 1 is a diagram illustrating a representative neuromuscular electrical stimulation (NMES) treatment;

FIG. 2A is an image of an NMES/EMG sleeve, according to an embodiment;

FIG. 2B is an image of the NMES/EMG sleeve of FIG. 2A as worn by a subject, according to an embodiment;

FIG. 3 is a flowchart diagram of a method for using EIT to determine necessary alignment changes following the donning process of an electrode garment 300, according to an embodiment; and

FIG. 4 is a is a flowchart diagram of a method for adjusting the original calibration parameters to accommodate the determined alignment variations, according to an embodiment.

### DETAILED DESCRIPTION OF THE PREFERRED EMBODIMENTS

Systems and methods leveraging electrical impedance tomography (EIT) to adjust electrode-based recording and/or stimulation calibrations that are dependent on electrode placement are also disclosed. These methods may be applied to garments designed for neuromuscular electrical stimulation (NMES) and/or electromyography (EMG) to ensure consistent electrode alignment and provide a method for autonomous recalibration. However, as will be appreciated by one having ordinary skill in the art, this method may be applied to any electrode-based recording and/or stimulation calibrations which are dependent upon electrode placement.

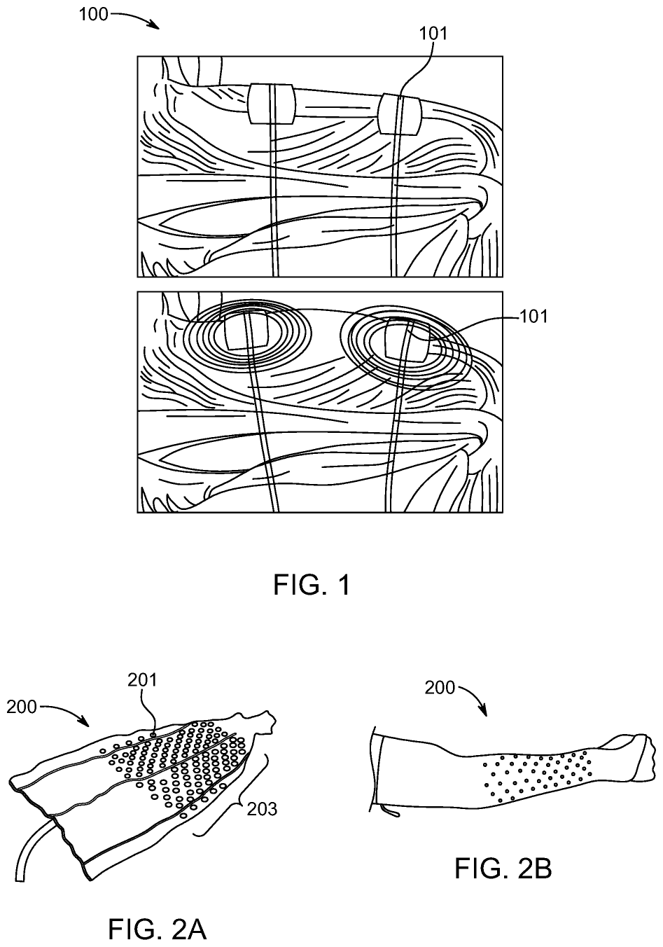

FIG. 1 is a diagram illustrating a representative NMES treatment 100. NMES comprises delivering electrical pulses via electrodes to skeletal muscles in order to activate a motor response. Muscle fibers in skeletal muscles respond to electrical signals sent through motor neurons. NMES induces a foreign electrical current which overrides the natural motor neuron activity and causes a muscle contraction. This is beneficial for individuals with impaired neuronal connections, such as spinal cord injury (SCI) or stroke patients. NMES may be used to achieve movement of paralyzed limbs. NMES may also be used to enhance movement of able limbs, for example, in sports performance enhancement and therapy. Functional electrical stimulation (FES) is a subset of NMES which focuses on promoting functional movement. In FIG. 1, electrodes 101 are placed on a subject's skin and activated, delivering electrical impulses to skeletal muscles and thereby causing a muscle contraction. A garment comprising an array of electrodes embedded therein may be configured to provide NMES treatments.

FIG. 2A is a sleeve-like NMES/EMG device 200 in an open position, according to an embodiment. FIG. 2B is an image of the NMES/EMG sleeve 200 as worn by a subject, according to an embodiment. The NMES/EMG sleeve 200 may comprise an array 203 of high density electrodes 201 which contact the skin of a subject to stimulate one or more muscles in the forearm and to record muscle activity. In some embodiments, a conductive medium, such as a hydrogel, may be placed between the electrode and the skin. In some embodiments, the electrodes 201 are relatively small to allow for fine motor control. In some embodiments, the NMES sleeve 200 may comprise as many as 160 electrodes 201. Each electrode 201 of the array of electrodes 203 may comprise an anode or a cathode.

Each electrode 201 of the array of electrodes 203 may be configured to be inactive or active. The active electrodes may be configured to be an anode (i.e., generate current) or to be a cathode (i.e., receive current). As used herein, the term “pattern” refers to the specific configuration of active and inactive electrodes, as well as the amplitude and waveform of each electrode.

Alternative devices for electrical stimulation include subcutaneous implantable neurostimulation devices. These implantable devices are wrapped around a target nerve and generally include one or more electrodes arranged to stimulate the nerve. By including more than one electrode and/or a different geometry of electrodes, implantable devices such as the flat interface nerve electrode (FINE) have been able to achieve stimulation selectivity at the level of individual nerve vesicles.

EIT is a noninvasive type of medical imaging in which the electrical conductivity of a part of the body is inferred from surface electrode measurements and used to form a tomographic image of that part. Specifically, EIT uses an array of surface electrodes and high frequency alternating current (AC) to measure internal electrical impedance. By placing an array of electrodes around a body part, it is possible to reconstruct the internal impedance distribution and infer the internal structure. For example, EIT measurements may be used to generate an anatomical model of a limb of interest and identify locations of rigid anatomical markers, such as bone. Systems and methods for generating an anatomical model of a limb are disclosed in the co-pending application titled “FINITE ELEMENT MODEL OF CURRENT DENSITY AND ELECTRICAL IMPEDANCE TOMOGRAPHY BASED METHOD FOR FUNCTIONAL ELECTRICAL STIMULATION”, which is incorporated by reference as if fully set forth.

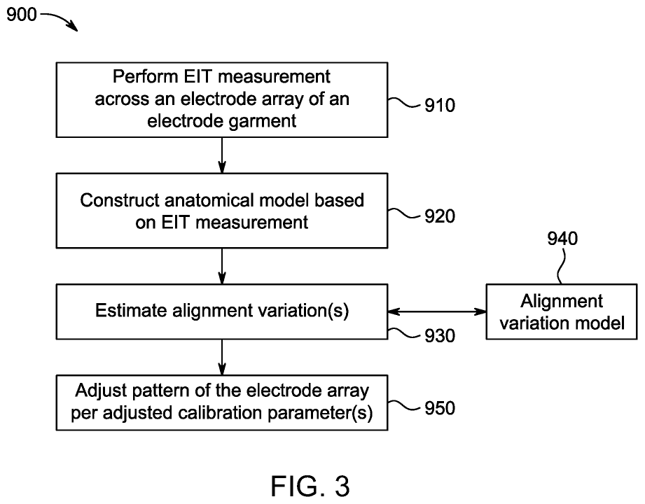

FIG. 3 is a flowchart diagram of a method for using EIT to determine necessary alignment changes following the donning process of an electrode garment 900, according to an embodiment.

At 910, following the donning of an electrode garment, such as a NMES/EMG sleeve, an EIT measurement will be made across the electrode array of the electrode garment. In some embodiments, the EIT measurement is a rapid EIT measurement across the electrode array of the electrode garment.

At 920, the EIT measurement may be used to generate an anatomical model of a limb of interest and identify locations of rigid anatomical markers, such as bone. In some embodiments, three-dimensional (3D) EIT may be used to construct a 3D anatomical model of the limb of interest. In some embodiments, the anatomical model may comprise a finite element model (FEM). The anatomical model may comprise a plurality of electrodes. In some embodiments, the electrodes of the anatomical model may mirror the placement of electrodes of the electrode garment. In some embodiments, the electrodes may be physically modeled as a circular disk with stainless steel material properties. However, as will be appreciated by one having ordinary skill in the art, the electrodes may be physically modeled as having different shapes and/or different material properties. Further, in some embodiments, the electrodes may be anchored flush to the skin surface of the model. In some embodiments, a conductive medium, such as a hydrogel may be placed between the electrode and the skin of the model. In alternative embodiments, the electrodes may be implanted in the anatomical model to mimic the effects of a subcutaneous implantable neurostimulation device. The electrodes may form an array of electrodes.

At 930, one or more alignment variations are estimated. The one or more alignment variations may indicate how much the electrode garment has shifted with respect to a reference alignment. By way of example, it may be determined that a distal shift of “x” mm occurred during the donning process. In some embodiments, the alignment variation may comprise one or more of a distal shift, a proximal shift, and/or a relative electrode distance to muscles in different sized arms.

The alignment variations may be estimated by an alignment variation model 940. The alignment variation model 940 may be based on previously collected data. In some embodiments, the alignment variation model 940 may comprise a shared response model. In other embodiments, the alignment variation model 940 may comprise a domain adaptation model. Both the shared response model and the domain adaption model may comprise two parts. In the first part, the determined electrode alignment is aligned to the reference alignment to determine one or more alignment variation(s). The alignment may comprise learning (i.e., estimating) a transformation function. However, as will be appreciated by one having ordinary skill in the art, the shared response model and the domain adaptation model take different approaches to estimating the transformation function. In the second part, a standard classifier or regression algorithm may be trained using collected alignment variation data.

Machine learning may be utilized to improve the alignment variation model 940 over time. In some embodiments, machine learning models which take input data and output predictions may be used. For example, machine learning techniques including, but not limited to, deep learning model, support vector machine, and linear or logistic regression, may be used. The machine learning may comprise a series of transformations in which the estimated alignment variation(s) are compared to a reference alignment over multiple iterations.

At 950, the original calibration parameters of the array of electrodes are automatically adjusted to new calibration parameters. In some embodiments, the pattern of the electrode array may be adjusted. Adjusting the pattern of the electrode may comprise adjusting one or more active electrodes of the electrode garment. For example, if it was determined that a distal shift of “x” mm occurred during the donning process, the alignment adjustment function may adjust the electrode pattern such that it shifted distally by “x” mm. In some embodiments, the one or more original calibration parameters may be adjusted using an alignment adjustment function, discussed in more detail with respect to FIG. 3.

In some embodiments, the method of FIG. 3 further comprises optimizing the electrode current, as disclosed in the co-pending application titled “FUNCTIONAL ELECTRICAL STIMULATION CALIBRATION SYSTEM, DEVICES AND METHODS”, which is incorporated by reference as if fully set forth.

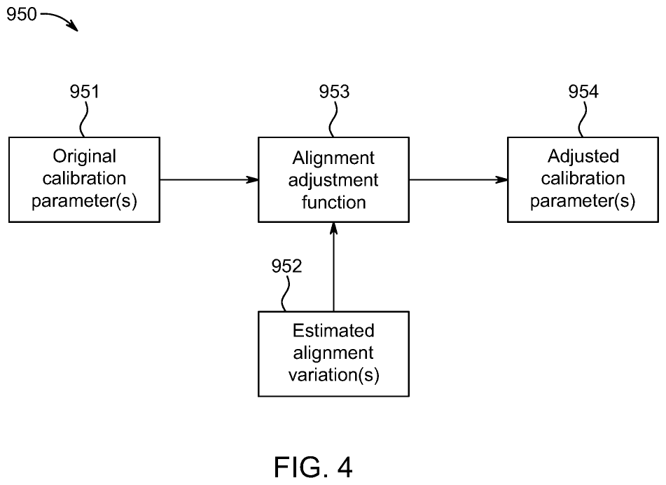

FIG. 4 is a flowchart diagram of a method for adjusting the original calibration parameters to accommodate the determined alignment variations 950, according to an embodiment. In some embodiments, the original calibration parameters 951 and the determined alignment variations 952 are input into the alignment adjustment function 953 to determine the adjusted calibration parameters 954. The electrode array of the electrode garment may be automatically adjusted according to the adjusted calibration parameters 954. Therefore, the stimulation pattern in a reference position that generates a desired muscle movement for a reference subject may be adjusted such that it may also generate the desired muscle movement for a new subject.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random-access memory (RAM), a register, cache memory, semiconductor memory devices, magnetic media such as internal hard disks and removable disks, magneto-optical media, and optical media such as CD-ROM disks, and digital versatile disks (DVDs).

It will be appreciated that the terminology used in the present application is for the purpose of describing particular embodiments and is not intended to limit the application. The singular forms “a”, “the”, and “the” may be intended to comprise a plurality of elements. The terms “including” and “comprising” are intended to include a non-exclusive inclusion. Although the present application is described in detail with reference to the foregoing embodiments, it will be appreciated that those foregoing embodiments may be modified, and such modifications do not deviate from the scope of the present application.

What is claimed is:

1. A method for calibrating an electrode array, the method comprising:
receiving an electrical impedance tomography (EIT) measurement from a plurality of electrodes contained in an electrode array;
generating an anatomical model of a limb based on a medical image of the limb;
determining an alignment of the electrode array with the anatomical model of the limb based on the EIT measurement;
comparing the alignment of the electrode array with a reference alignment; and
estimating one or more alignment variations using an alignment variation model, wherein the one or more alignment variations are used to calibrate the electrode array.

2. The method of claim 1, wherein the anatomical model is a finite element model.

3. The method of claim 1, wherein the medical image is an EIT.

4. The method of claim 1, further comprising automatically adjusting a pattern of the electrode array to accommodate the one or more alignment variations using an alignment adjustment function.

5. The method of claim 4, wherein automatically adjusting the pattern of the electrode array comprises sending one or more signals to the electrode array to shift a pattern of active electrodes and inactive electrodes.

6. The method of claim 1, further comprising manually adjusting a pattern of active electrodes and inactive electrodes of the electrode array to accommodate the one or more alignment variations using an alignment adjustment function.

7. The method of claim 1, wherein the limb is a forearm.

8. The method of claim 1, wherein the electrode array is located on an internal surface of a garment.

9. The method of claim 8, wherein the method is performed following a donning of the garment.

10. The method of claim 1, wherein the alignment variation model is a shared response model or a domain adaptation model.

11. The method of claim 1, wherein machine learning is used to improve alignment variation model.

12. The method of claim 11, wherein the machine learning comprises a deep learning model, support vector machine, or linear or logical regression.

13. The method of claim 1, wherein the one or more alignment variations comprise one or more of a distal shift, a proximal shift, or a relative electrode distance to muscles in different sized arms.

14. The method of claim 1, further comprising optimizing the electrode current of the electrode array.

15. A system comprising:
an electrode array comprising a plurality of electrodes, the electrode array configured to perform an electrical impedance tomography (EIT) measurement; and
a processor communicatively coupled to the electrode array, the processor configured to:
construct an anatomical model of a limb based on a medical image of the limb,
determine an electrode alignment of the electrode array with the anatomical model of the limb based on the EIT measurement,
compare the alignment of the electrode array with a reference alignment; and
estimate one or more alignment variations using an alignment variation model, wherein the one or more alignment variations are used to calibrate the electrode array.

16. The system of claim 15, wherein the electrode array is located on an internal surface of a garment.

17. The system of claim 16, wherein the garment is a sleeve configured to be worn on a forearm.

18. The system of claim 15, wherein the electrode array is a high density electrode array.

19. The system of claim 15, wherein the electrode array is configured to perform functional electrical stimulation (FES), electromyography (EMG), or both FES and EMG.

20. The system of claim 15, wherein the processor is further configured to automatically adjust a pattern of the electrode array to accommodate the one or more alignment variations using an alignment adjustment function.
